(19) United States
(12) Patent Application Publication
(10) Pub. No.: US 2014/0275827 A1
(43) Pub. Date: Sep. 18, 2014

(54) METHOD AND SYSTEM FOR DERIVING EFFECTIVENESS OF MEDICAL TREATMENT OF A PATIENT

(71) Applicant: PACESETTER, INC., Sylmar, CA (US)

(72) Inventors: Amreeta Gill, Los Angeles, CA (US); Tyler MacBroom, Los Angeles, CA (US); Sergio Shkurovich, Encino, CA (US)

(73) Assignee: PACESETTER, INC., Sylmar, CA (US)

(21) Appl. No.: 13/895,083

(22) Filed: May 15, 2013

Related U.S. Application Data
(60) Provisional application No. 61/781,882, filed on Mar. 14, 2013.

(51) Int. Cl. A61B 5/00

(52) U.S. Cl. CPC A61B 5/4848
USPC 600/301; 600/300; 128/898; 607/5; 607/18; 607/62

(57) ABSTRACT

A method and system for deriving effectiveness of medical treatment of a patient are provided that include collecting patient state (PS) data from at least one of an implantable medical device (IMD) or an external medical device (EMD) over a collection interval. The collected PS data relates to a physiologic characteristic (PC) of the patient. The PS data is transferred to a database that is remote from the patient to form a patient state data (PSD) history. The patient undergoes a pivotal medical event (PME) during the collection interval. The PS data within the PSD history is analyzed before and after the PME to propose a treatment therapy (TT). Following delivery of the TT, the collecting and transferring operations are repeated to obtain post-treatment PS data and form a post-treatment PSD history. An effectiveness indicator (EI) of the TT is derived based on at least the post-treatment PSD history.

### CROSS REFERENCE TO RELATED APPLICATIONS

This application claims the benefit of U.S. Provisional Application Ser. No. 61/781,882, filed Mar. 14, 2013.

### BACKGROUND OF THE INVENTION

Embodiments of the present invention generally relate to patient medical care management, and more specifically to methods and systems for deriving effectiveness of medical treatment.

Currently, a large portion of the work in disease management, including screening, diagnosis, and treatment of diseases, is done without the assistance of technology. Today, physicians review the progression of a particular disease through multiple stages. Physicians may have to personally compile data from multiple sources in order to make an informed diagnosis and treatment recommendation.

A need remains for an effective way to allow multiple implantable and external medical devices to communicate with each other to support an integrated approach to disease management, across the dimensions of stage and state, supported by technology to alleviate some of the burden from physicians.

### SUMMARY

As medical technology advances, patients are increasingly likely to benefit from multiple external and/or implanted medical devices during the course of their treatment. For example, the screening and treatment of each disease state and at each stage of progression may employ one or more different external and/or implantable medical devices. In addition, human anatomy may require that implantable medical devices that would ideally be integrated into one device be split into two in order to reside in different locations in the body. Typically, each of the medical devices acts independently of any other medical devices implanted within the body or external to the body.

In accordance with one embodiment, a method is provided for deriving effectiveness of medical treatment of a patient. The method includes collecting patient state (PS) data from at least one of an implantable medical device (IMD) or an external medical device (EMD) over a collection interval. The at least one IMD or EMD is configured to collect the PS data in relation to a physiologic characteristic (PC) of the patient. The method also transfers the PS data to a database that is remote from the patient to form a patient state data (PSD) history. The patient undergoes a pivotal medical event (PME) during the collection interval. Additionally, the method includes analyzing the PS data within the PSD history before and after the PME to propose a treatment therapy (TT). Following delivery of the TT, the method repeats the collecting and transferring operations to obtain post-treatment PS data and form a post-treatment PSD history. The method further includes deriving an effectiveness indicator (EI) of the TT based on at least the post-treatment PSD history.

Optionally, the method for deriving effectiveness of medical treatment of a patient further includes adjusting the TT based on the post-treatment PSD history. Adjusting the TT may include a prescription change or an acute intervention. Adjusting of the TT may include changing an output of the at least one IMD or EMD. Deriving the effectiveness of the TT may include comparing PS data that was collected from at least two different types of IMD or EMD before and after the PME. The collecting may include collecting first PS data from a first IMD or EMD and collecting second PS data from a second IMD or EMD. The PME may represent at least one of a clinical event, an acute intervention, or a prescription change. The acute intervention may include an ablation, implantation of an implantable cardioverter-defibrillator (ICD) device, left atrial appendage (LAA) closure, cardiac resynchronization therapy (CRT), spinal cord stimulation (SCS) therapy, renal denervation, or deep brain stimulation (DBS) therapy. The clinical event may include at least one of a heart attack, a stroke, or detection of atrial fibrillation (AF), heart failure, or hypertension.

In accordance with one embodiment, a method is provided for deriving effectiveness of medical treatment of a patient. The method includes collecting first patient state (PS) data from a first one of an implantable medical device (IMD) or an external medical device (EMD) over a collection interval. The first IMD or EMD is configured to collect the first PS data in relation to a first physiologic characteristic (PC) of the patient. The method also includes collecting second PS data from a second one of an IMD or EMD. The second IMD or EMD is configured to collect the second PS data in relation to a second PC of the patient. The first PC differs from the second PC. The method further includes transferring the first and second PS data to a database that is remote from the patient to form a patient state data (PSD) history. The patient undergoes a treatment therapy (TT) during the collection interval. Additionally, the method includes deriving an effectiveness indicator (EI) of the TT, by an assessment module, based on the first and second PS data collected before and after the TT.

In accordance with one embodiment, a system is provided for deriving effectiveness of medical treatment of a patient. The system includes at least one of an implantable medical device (IMD) or an external medical device (EMD) for collecting patient state (PS) data from over a collection interval. The at least one IMD or EMD is configured to collect the PS data in relation to a physiologic characteristic (PC) of the patient. The system also includes a database configured to receive the PS data to form a patient state data (PSD) history. The database is remote from the patient. The patient undergoes a pivotal medical event (PME) during the collection interval. The system further includes an analysis module configured to analyze the PS data within the PSD history before and after the PME to propose a treatment therapy (TT). Following delivery of the TT, the IMD or EMD and database repeat the collecting and receiving operations to obtain post-treatment PS data and a post-treatment PSD history. The system also includes an assessment module configured to derive an effectiveness indicator (EI) of the TT based on at least the post-treatment PSD history.

In accordance with one embodiment, a system is provided for deriving effectiveness of medical treatment of a patient. The system includes a first one of an implantable medical device (IMD) or an external medical device (EMD) configured to collect first patient state (PS) data over a collection interval. The first IMD or EMD is configured to collect the first PS data in relation to a first physiologic characteristic (PC) of the patient. The system includes a second one of an IMD or EMD configured to collect second PS data in relation to a second PC of the patient. The first PC differs from the second PC. Additionally, the system includes a database located remote from the patient. The database is configured to receive the first and second PS data to form a patient state data (PSD) history. The patient undergoes a treatment therapy (TT) during the collection interval. The system further includes an assessment module configured to derive an effectiveness indicator (EI) of the TT based on the first and second PS data collected before and after the TT.

### BRIEF DESCRIPTION OF THE DRAWINGS

FIG. 1 is a flow chart showing a medical treatment management process according to an embodiment.

FIG. 2 illustrates a block diagram of a medical treatment management system according to one embodiment.

FIG. 3 is a diagram of a medical treatment management system implemented according to an embodiment.

FIG. 4 illustrates a distributed processing system in accordance with one embodiment.

FIG. 5 is a diagram showing an example medical treatment management system applied to a patient with atrial fibrillation (AF).

FIG. 6 is a diagram showing an example medical treatment management system applied to a patient with AF.

FIG. 7 is a flow chart for a medical treatment management process for a patient diagnosed with AF.

FIG. 8 is a diagram showing an example medical treatment management system applied to a patient with chronic and acute hypertension.

FIG. 9 is a map of a medical treatment management system according to an embodiment that is scalable to integrate various types of data from various sources.

FIG. 10 is a diagram showing an example medical treatment management system applied to a patient with chronic and acute pain.

FIG. 11 illustrates a flowchart of one embodiment of a process for deriving the effectiveness of medical treatment of a patient.

FIG. 12 illustrates a flowchart of another embodiment of a process for deriving the effectiveness of medical treatment of a patient.

### DETAILED DESCRIPTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware and circuitry. Thus, for example, one or more of the functional blocks (e.g., processors or memories) may be implemented in a single piece of hardware (e.g., a general purpose signal processor or a block or random access memory, hard disk, or the like). Similarly, the programs may be stand alone programs, may be incorporated as subroutines in an operating system, may be functions in an installed imaging software package, and the like. It should be understood that the various embodiments are not limited to the arrangements and instrumentality shown in the drawings.

FIG. 1 is a flow chart showing a medical treatment management process 100 according to an embodiment. The medical treatment management process 100 may represent a comprehensive disease management solution that includes screening, diagnosis, and treatment of major diseases. At 102, the process 100 monitors a patient to screen for a medical condition and/or event. For example, the patient may be monitored for a condition such as coronary artery disease (CAD) and/or an event such as a myocardial infarction (MI). The monitoring at 102 may be performed by, for example, one or more implantable medical devices (IMD), one or more external medical devices (EMD), or a combination of both, that are designed to sense and collect patient state (PS) data relating to one or more physiologic characteristics (PC) of the patient.

After monitoring, a diagnosis is made at 104 based on the data collected during monitoring. The diagnosis 104 may indicate the presence or absence of a medical condition/event in the patient. Depending on the diagnosis 104, the process 100 may proceed to 106 where no additional action is taken at the present time. In such a case, the process 100 returns to 102 where the patient is continuously monitored, and the process 100 repeats.

Referring back to the diagnosis 104, the process 100 may alternatively proceed to 108, where a course of action is recommended. Two potential actions in response to a diagnosis 104 are an acute intervention at 110 and chronic management at 112. At 110, an acute intervention may be a specific medical procedure, such as an ablation, implantation of an implantable cardioverter-defibrillator (ICD) device, left atrial appendage (LAA) closure, cardiac resynchronization therapy (CRT), spinal cord stimulation (SCS) therapy, renal denervation, deep brain stimulation (DBS), and the like. Alternatively, examples of chronic management at 112 may include a prescription change, physical therapy, a change in diet and/or exercise, and other non-surgical events. Optionally, some actions may require an acute intervention and also a chronic management change. In either case, after the acute intervention at 110 or the chronic management change at 112, the process 100 returns to the monitoring step at 102.

The process 100 iteratively monitors the same PCs of the patient to determine how the action taken has affected the PS data by comparing the post-action PS data to the pre-action PS data. This comparison may affect the subsequent diagnosis at 104 and determine whether further actions are to be taken at 108. Therefore, the process 100 may be cyclical and may be performed continuously to track the patient's care management over time.

The process 100 may be performed by a comprehensive (remote care) system that integrates data received from multiple sources, such as multiple IMDs and/or EMDs, to improve patient care. For example, the multiple sources may achieve communication with each other, with intermediary equipment, with external servers, and the like. The interaction between devices may provide an integrated approach to patient care management, and alleviate some of the burden from physicians to integrate information and provide an informed diagnosis and recommendation for action.

FIG. 2 illustrates a block diagram of a medical treatment management system 200 according to one embodiment. The system 200 may be used to perform the medical treatment management process 100 shown in FIG. 1. In the illustrated embodiment, a patient 202 has two IMDs, 204 and 206, implanted within the body and one EMD 208 external to the body. Optionally, the patient 202 may have any number of IMDs and EMDs. The IMDs 204, 206 and EMD 208 are each configured to sense and monitor a PC of the patient 202 to obtain PS data relating to the PC. In addition, the IMDs 204, 206 and/or EMD 208 may be configured to deliver treatment to the patient 202, such as SCS, DBS, and/or cardiac stimulation.

The system 200 also includes a database 210, which may be a server 210, or housed within the server 210. The PS data collected by the IMDs 204, 206 and EMD 208 are sent to the server 210, located remotely from the patient 202, where the PS data may be integrated, processed, and/or stored. The server 210 may store received PS data from each of the IMDs/EMDs 204-208 over time, creating a patient state data (PSD) history. The server 210 may include an analysis module (not shown) that is configured to analyze the PS data within the PSD history before and after the PME to propose a treatment therapy (TT). The server 210 may also include an assessment module (not shown) configured to derive an effectiveness indicator (EI) of the TT based on at least the post-treatment PSD history. For example, the EI may be derived based on the first and second PS data collected before and after the TT, such as by comparing the pre-treatment first and second PS data to the post-treatment first and second PS data. The assessment module may be configured to propose a potential prescription change to a user based on the assessment of the effectiveness of the TT. Optionally, the assessment module may set operating parameters of the first IMD or EMD and operating parameters of the second IMD or EMD, both based at least in part on the first PS data and the EI.

The server 210 may also include a treatment therapy (TT) control module (not shown). The TT control module is configured to adjust the treatment therapy based at least on the post-treatment PSD history. The TT control module may adjust the treatment therapy by changing a prescription or an acute intervention. Alternatively or in addition, The TT control module may be configured to adjust the treatment therapy by changing an output of the at least one IMD or EMD. For example, the TT control module may be configured to change at least one of a sensing, programmed parameters, and an output of the first IMD or EMD based on the second PS data from the second IMD or EMD. The TT control module may be configured to coordinate functional operation of the first IMD or EMD and the second IMD or EMD based on the EI and the first and second PS data.

The server 210 may be accessed using a website graphical user interface (GUI) 212. An example of a website GUI 212 is the Merlin.net™ Patient Care Network. Therefore, the server 210 may be accessed remotely using an internet or other network connection through the website GUI 212. For example, a clinician 214 may use the website GUI 212 to access the PS data that is in the server 210. The clinician 214, therefore, has the ability to access up-to-date PS data on a patient 202 that may be located remotely from the clinician 214, such as if the clinician 214 is at a medical hospital or clinic and the patient 202 is at home or even traveling out of the state/country. Optionally, the website GUI 212 may be used to send all or a selected amount of PS data to storage in an electronic medical/health record (EMR/EHR) system 216, such as the record systems commonly operated by hospitals and clinics to track the medical history of its patients.

In addition to the clinician 214 having the ability to access patient information through the website GUI 212, the website GUI 212, or a software/hardware extension thereof, may be configured to contact the clinician 214. For example, if the PS data collected by the server 210 indicates that the patient 202 is experiencing an emergency medical event, such as an MI, this information may be communicated from the server 210 to the website GUI 212, where an alert is then sent to notify emergency medical services and/or the clinician 214. For example, the alert may be sent to the phone of the clinician 214 as an SMS text message or automated call.

In an embodiment, the server 210 may also be configured to communicate with the patient 202 through a patient communication module 218 that is accessible to the patient 202. The server 210 (and database) may be directly connected to the patient communication module 218 or connected through the website GUI 212. The patient communication module 218 may include one or more of a phone, a tablet, a computer, a patient advisory module (PAM), and a patient facing interface (such as in a hospital room), and the communications may be relayed via email, fax, SMS message, software application and/or program, and the like. For example, an alert may be sent to the patient 202 through the patient communication module 218 to notify the patient 202 of a medical event that the patient 202 may not be aware of, such as a silent MI. The patient 203 not only may receive information through the patient communication module 218, but also the patient 202 may use the module 218 to communicate with the server 210. For example, the patient 202 may use the module 218 to indicate to the system 200 that the patient is experiencing pain or another physiologic symptom and to rate the amount of pain. This input information from the patient 202 may be sent to the server 210 another source of PS data to be integrated with the PS data collected from the IMDs 204, 206 and the EMD 208.

The system 200 may also optionally include intermediary equipment operatively connected between the medical devices 204-208 on the patient 202 and the server 210. For example, the illustrated embodiment includes intermediary equipment 220 and 222. The intermediary equipment 220 and 222 is configured to enhance communicative capabilities in the system 200, and to provide communicative options. For example, the IMDs 204, 206 and EMD 208 may be configured to communicate with each other through various methods, such as through RF, Wi-Fi, wired connection, and ultrasonic communication, among others. Communicative options include communication directly between devices 204-208, indirectly through intermediary equipment 220 and/or 222, or indirectly through the server 210 (with or without the intermediary equipment 220, 222 in the system 200). Intermediary equipment 220, 222 may be configured to communicate with each other directly or via the server 210. Communication, either device-to-device or device-to-intermediary equipment, may be achieved through a physical connection, inductive telemetry, or RF telemetry. Connectivity between intermediary equipment 220, 222 may be achieved through phone lines, cellular connectivity, Wi-Fi connectivity, or wired Internet connectivity.

The intermediary equipment 220, 222 may optionally be servers that have some data storage and/or analyzation capabilities. Apart from increasing communication options and decreasing path lengths between the server 210 and the devices 204-208, the intermediary equipment 220, 222 may make up for some technical limitations in the devices 204-208. For example, the intermediary equipment 220, 222 may have additional power, better radios, and more processing space than the devices 204-208, which allows the devices 204-208 to be designed smaller and lighter, both characteristics that are beneficial for IMDs 204, 206 in particular, since they are implanted within a patient 202. One example of intermediary equipment is a Merlin™@home transmitter.

The system 200 may be used to carry out the cyclical process 100 described in FIG. 1. For example, the monitoring at 102 may be performed by the IMDs 204, 206, and EMD 208, and the PS data collected transmitted to the server 210. The server 210 may include a processor (not shown) that integrates the received PS data and uses the data to make a diagnosis, as at 104. Whether the diagnosis at 104 leads to further action taken 108 or not 106, the process 102 returns to the monitor step at 104. Thus, in the system 200, the IMDs 204, 206, and EMD 208 continuously monitor the PCs of the patient, and the collected PS data is used to make comparisons and recommendations, with or without the input of a clinician.

In an example embodiment, the system 200 may receive information from a few inputs, and may make a variety of comparisons, recommendations, and other outputs based on the input information. For example, the inputs may be PS data acquired by the IMDs 204, 206, and/or EMD 208 (i.e., device data), subjective PS data input by the patient 202 using a patient communication module 218, pivotal medical events of the patient 202, and/or external data acquired from a database including medical records from a large population of patients, such as from pharmacies and insurance companies. As used herein, a “pivotal medical event” is a key medical event in a patient's medical history and may be either a clinical event, such as a heart attack or stroke, or a human intervention, such as a medication change or an acute intervention.

The system 200 receives the input information at the server 210, integrates and processes the information, and may take one or more output steps in response. For example, the system 200 may create a trend or graph that integrates data from multiple devices together or displays PS data from one device over time. The system 200 may compare device data recorded before and after a pivotal medical event transpired. By comparing integrated device data, the system 200 may assess the effectiveness of an acute or chronic treatment. The system 200 may provide automatic feedback or programming to one or more medical devices (which may or may not be one of the IMDs 204, 206, or EMD 208). As mentioned above, the system 200 may alert the physician 214 to intervene in patient care. For example, the system 200 may recommend that the physician/clinician 214 (i) have the patient 202 call or visit the clinic, (ii) change a prescription, (iii) program/adjust a device, (iv) gather more tests/data before making a diagnosis, and/or (v) perform an acute procedure on the patient 202, among other potential recommendations. Optionally, the system 200 may be configured to use the input information to automatically change a prescription, without the intervention of a clinician 214 (e.g., if the change is to lower the dosage of the prescription).

In one example, the input to the system 200 comes from one or more IMDs 204 and/or 206, such as ICDs, pacemakers, or NS devices. The IMDs 204, 206 communicate with each other, with EMDs, and with intermediary equipment 220 and/or 222 wirelessly through RF. The intermediary equipment 220/222 communicates with the server 210 through an analogue phone line. The output of the system 200 are diagnostic results, which are accessible via website, smartphone app, native computer program, and/or print outs.

In another example, the input to the system 200 comes from one or more EMDs 208, such as a blood pressure cuff or a scale. The EMD 208 communicates with other EMDs/IMDs and/or intermediary equipment 220/222 wirelessly through inductive telemetry. The intermediary equipment 220/222 communicates with the server 210 through the Internet. The output of the system 200 is a trend or graph, which is viewable using a website, a native computer program, and/or a smartphone app.

In a further example, an external database is the input to the system 200. The external database may be an insurance database, a pharmacy database, and/or EMR systems 216. The external database communicates with the EMDs 208 and the intermediary equipment 220/222 through a wired connection. The intermediary equipment 220/222 communicates with the server 210 wirelessly through Wi-Fi. The output of the system 200 may be an alert, which is sent to a clinician 214 via email, phone call, fax, and/or SMS message.

Another example includes an interactive voice response (IVR) phone system as an input. The IVR phone system uses a touchtone or voice-based input to provide information to the system 200. The patient 202 may use the IVR phone system to input subjective data and/or PS data relating to monitored PCs, or to receive information from the system 200. The intermediary equipment 220/222 communicates with the server 210 through a cellular network. The output of the system 200 is a treatment effectiveness indicator/measurement. The output is implemented by displaying data (e.g., numbers, tables, trends) before and after the treatment (or modification of the treatment). Statistics such as T-tests may accompany the display data. Statistical analysis may form the basis of recommendations made by the system 200.

A website may be an input in another example, as the system 200 may receive input information that was sent using a website. The website communicates with the server 210 and/or other components of the system 200 through a cable network. The output of the system 200 is a recommendation to a clinician 214. For example, the clinician 214 may log in to the website, sending log-in information to the system 200, to receive the recommendation. The output to the clinician 214 may also be implemented through email, SMS message, and/or native computer program to the clinician 214.

In another example, a computer program is the input, as the computer program is used to input information to the server 210. The computer program communicates with the server 210 through a digital phone network. The output of the system 200 is a prescription change. The prescription change may be accomplished with or without physician approval. The prescription change may be done manually through SMS message, email, fax, and/or phone. Alternatively, the prescription change may be automated by the system 200 through the patient communication module 218, which communicates with the patient via SMS, email, fax, phone, patient facing website, patient facing mobile app, and/or patient facing device (e.g., PAM).

Another input to the system 200 may be an internal database within the immediate hospital, clinic, research institute, and the like. In an example, the output by the system 200 is device programming. Device programming may change sensing, programmed parameters, and/or output of one or more devices (e.g., 204, 206, and/or 208) based on the data from another device.

The system 200 may be configured to support additional inputs. One example is a pivotal medical event (PME). A PME may include a clinical or biological event such as myocardial infarction (MI), decompensation, stroke, and the like. Besides clinical events, a PME may also represent acute interventions, such as implantation of an IMD or other procedures, and prescription changes (discussed as an input above). For example, the system 200 may record information about the PME, such as the nature of the PME, the date, observations by the clinician 214, and other details. Another example of an input is medical equipment at clinics and/or hospitals, such as ablation systems and cardiac mapping systems. This medical equipment may be configured to communicate with and transfer collected PS data to the system 200 automatically or in response to user intervention. Another example input is a user facing device. The user facing device may be a workstation that faces the clinician 214, or a PAM (for example) that faces the patient 202. Using the user facing device, the clinician 214 may enter notes and observations into the system 200 to be stored in the server 210, and/or the patient 202 may enter information, such as subjective pain levels and/or a log of daily food intake or activity.

In an example application of a few system 200 inputs and outputs, the system 200 is used with a patient 202 that has heart failure (HF) and has had three IMDs 204/206, a cardiac resynchronization therapy (CRT) device, a left atrial pressure (LAP) monitor, and a spinal cord stimulation (SCS) device, implanted within the body of the patient 202 to monitor and treat the HF. Each night, the CRT device connects to intermediary equipment 220/224 and requests permission form the server 210 to temporarily disable the SCS device in order to make accurate impedance measurements. The server 210 analyzes the LAP data collected from the LAP monitor that is stored in the internal database within the server 210, and decides that it is alright to disable the SCS device for a few minutes. The next morning, the patient facing device 218 sounds an alarm for the patient 202 to take his/her prescription HF medications. The prescription regimen of the patient 202 was previously input by the patient's cardiologist 214 using a physician facing website. The patient 202 takes the medication and enters into the patient facing device 218 that the medicine was taken to confirm compliance.

FIG. 3 is a diagram of a medical treatment management system 300 implemented according to an embodiment. The system 300 may be the medical treatment management system 200. FIG. 3 illustrates some of the various IMDs and EMDs that may be functionally integrated within the system 300. The system 300 has the interoperability to communicate with products designed for different medical conditions, such as cardiovascular devices, cardiac rhythm management devices, atrial fibrillation devices, neuromodulation devices, and the like. For example, a blood pressure (BP) cuff 302 may be an EMD that is used to test for high blood pressure, a sign of hypertension. Blood pressure measurements may be used to screen for renal denervation candidates, and may also be used to monitor the success of a renal denervation acute intervention after the fact.

The system 300 may also integrate PS data received from cardiac rhythm management devices 304, such as pacemakers and implantable cardioverter-defibrillators (ICDs). Additionally, atrial fibrillation (AF) sensing devices 306 other than pacemakers and ICDs may be integrated within the system 300. To treat AF, the devices 304 and 306 may be used to treat chronic AF in a patient, to screen for ideal candidates for an acute intervention (e.g., an ablation), and to monitor the post-operational success of the procedure, which may result in removal from medication.

Furthermore, the system 300 may integrate neuromodulation devices 308 designed for use in pain management and tremor control. The devices 308 may be IMDs configured to provide neurostimulation (NS), such as with SCS and/or DBS. A patient's heart failure condition may be treated and improved using SCS methods. In another example, the use of the stimulation devices with an accelerometer (as an EMD that may be worn as a wrist watch) may be used by the system 300 to monitor the success of DBS for tremor control. For pain management, the PAM 310 may be used by the patient to enter a subjective pain value on a pain scale, which may then be used by the clinician to determine whether the stimulation devices should be reprogrammed and/or whether the stimulation leads should be repositioned.

In the illustrated embodiment, the PS data may be sent from the devices 302-308 to intermediary equipment 312, which then transmits the PS data to a server for data storage. The PAM 310 may also be used to transmit PS data to the server. For example, the system 300 server may be a server within Merlin.net, which is then accessible to the patient and clinician through the website GUI 314 shown in FIG. 3.

FIG. 4 illustrates a distributed processing system 400 in accordance with one embodiment. The distributed processing system 400 includes a server 402 connected to a database 404, a programmer 406, a local RF transceiver 408, and a user workstation 410 electrically connected to a communication system 412. The local RF transceiver 408 may be at least a part of the intermediary equipment 220, 222 shown in FIG. 2. The local RF transceiver 408 may be operatively connected to at least one IMD 418 and/or EMD 420. The programmer 206 may be a user device, such as a patient or physician hand-held device. The programmer 406 may also be operatively connected to at least one IMD 418 and/or EMD 420, and configured to program and reprogram the IMD 418 and/or EMD 420. Although shown in FIG. 4 as separate entities, the IMD 418 and EMD 420 connected to the local RF transceiver 408 may be the same IMD 418 and EMD 420 connected to the programmer 406.

The communication system 412 may be the internet, a voice over IP (VoIP) gateway, a local plain old telephone service (POTS) such as a public switched telephone network (PSTN), a cellular phone based network, and the like. The communication system 412 may be integrated for use with additional communication methods, such as communication methods not yet in application. Alternatively, the communication system 412 may be a local area network (LAN), a campus area network (CAN), a metropolitan area network (MAN), or a wide area network (WAN). The communication system 412 serves to provide a network that facilitates the transfer/receipt of information such as ST segment changes, heart rate, blood pressure, and other PS data.

The server 402 is a computer system that provides services to other computing systems over a computer network. The server 402 controls the communication of information between devices on the network. The server 402 interfaces with the communication system 412 to transfer information between the programmer 406, the local RF transceiver 408, the IMD 418, the EMD 420, the user workstation 410, as well as a cell phone 414 and a personal data assistant (PDA) 416. The transferred information is sent to the database 404 for storage/retrieval of records or information. For example, the database 404 may be the EMR/EHR system 216 shown in FIG. 2. On the other hand, the server 402 may upload PS data directly from the IMD 418 and/or EMD 420 via the local RF transceiver 408 or the programmer 406.

The database 404 stores PS information such as ST segment changes, heart rates, blood pressure, blood glucose, weight, subjective pain ratings, and the like, for a single or multiple patients. The system 400 has the capability of pooling large volumes of data from multiple patients for data mining and analysis purposes, which may be used for predictive diagnostics and/or patient screening. The information is downloaded into the database 404 via the server 402 or, alternatively, the information is uploaded to the server from the database 404. The programmer 406 may reside in a patient's home, a hospital, or a physician's office. The programmer 406 may wirelessly communicate with the IMD 418 and/or EMD 420 and utilize protocols, such as Bluetooth, GSM, infrared wireless LANs, HIPERLAN, 3G, satellite, as well as circuit and packet data protocols, and the like. Alternatively, a hard-wired connection may be used to connect the programmer 406 to the IMD 418 and/or EMD 420. The programmer 406 may be used by a clinician to adjust the parameters of the IMD 418 and/or EMD 420, such as in response to a changed diagnosis. For example, with an IMD 418 that is used to provide SCS, the programmer 406 may adjust the intensity of the neurostimulation, such as to lower the intensity if monitoring of the patient has indicated that the patient's pain therapy has been successful and pain levels have decreased.

The local RF transceiver 408 interfaces with the communication system 412 to upload the PS data collected by the IMD 418 and EMD 420 to the server 402. In one embodiment, the IMD 418 and the EMD 420 have a bi-directional connection 424 with the local RF transceiver 408 via a wireless connection. The local RF transceiver 408 is able to acquire collected PS data from the IMD 418 and the EMD 420, and, conversely, the local RF transceiver 408 may download stored operating parameters from the database 404 to the IMD 418 and the EMD 420.

The user workstation 410 may interface with the communication system 412 via the internet or POTS. The user workstation 410 may be accessible to the patient, clinician, and other admitted persons. For example, the user workstation 410 may be a PAM, such as the PAM 310 shown in FIG. 3. The user workstation 410 may download the patient information and notifications to the cell phone 414, the PDA 416, the local RF transceiver 408, the programmer 406, or to the server 402 to be stored on the database 404. For example, the user workstation 410 may communicate data to the cell phone 414 or PDA 416 via a wireless communication link 426.

FIG. 5 is a diagram showing an example medical treatment management system 500 applied to a patient 502 with atrial fibrillation (AF). The medical treatment management system 500 may be the system 200 described in FIG. 2. In this example, a patient 502 feels palpitations, so schedules a clinic visit with a physician 504. Based in part on the palpitations, the physician 504 may recommend a procedure to implant an IMD 506 (or position an EMD) to monitor PC related to AF to test for AF. The IMD 506 may be one or more various sensors designed for cardiac rhythm management or AF, such as pacemakers, ICDs, and cardiac monitors, as any of these devices 506 may be used as a sensor for AF. Once the IMD 506 is implanted (or EMD is positioned), the device 506 collects PS data that is transferred to intermediary equipment 508 which transmits the data to a server 510 for storage and processing. The PS data may relate to the AF burden or other cardiac PCs of the patient 502. The server 510 is accessible to the physician 504 at the clinic, such as via a website GUI, so the physician 504 may review the PS data collected by the IMD 506 over time. Using the PS data, the physician 504 may, for example, diagnose the patient 502 with paroxysmal AF. The next step may be for the clinic to schedule an office visit with the patient 502 to discuss the diagnosis and potential responsive actions, such as a prescription drug therapy and/or an ablation procedure.

FIG. 6 is another diagram showing an example medical treatment management system 600 applied to a patient 602 with AF. The medical treatment management system 600 may be the system 200 described in FIG. 2. The patient 602 may have an ICD 604 that monitors his/her AF burden. Alternatively, or in addition, the patient 602 may use a home international normalized ratio (INR) monitor as an EMD, which measures the clotting tendency of the blood. The ICD 604 collects PS data on the patient's AF burden, and intermediary equipment 606 transmits the PS data to a server 608. The AF burden of the patient 602 is stored in the server 608 over time to create a PSD history for the patient 602. Alternatively, or in addition, the patient 602 may use a home international normalized ratio (INR) monitor, which measures the clotting tendency of the blood, as an EMD which communicates with the system 600.

In the example, the PS data collected by the ICD 604 indicates that the patient 602 has persistent AF. The system 600 recommends the acute intervention of an ablation based on the received PS data. A clinician (not shown) performs an ablation on the patient 602 using an ablation catheter 610, and the pivotal medical event of receiving an ablation is automatically marked within the system 600 (i.e., without the need for manually inputting the event). In addition, the clinician may provide a prescription (not shown) to the patient 602, such as an anti-coagulant medication, during the recovery period after the ablation procedure.

The system 600 may create a stroke risk profile for the patient 602 and recommend intervention at a certain risk level. For example, if a stroke is detected by a change in brain electrical activity, the system 600 may be configured to notify emergency medical services, the patient's family and the patient's physician.

After receiving the ablation, the post-ablation AF burden of the patient 602 is tracked using the implanted ICD 604. Thus, the system 600 operates cyclically. The post-ablation PS data relating to the AF burden is compared to the AF burden PS data recorded prior to the ablation procedure. This comparison shows the change in AF burden, and is used to indicate the effectiveness of the ablation procedure. If, for example, the data stored in the server 608 shows that the ablation has been successful in reducing the patient's AF burden, the system 600 may recommend the physician to reduce and/or stop the patient's anti-coagulant medication. The system 600, directly or through the physician, may “close the loop” (e.g., from diagnosis to treatment) by notifying the patient 602 to stop taking the medication. The patient 602 may be notified through one of a number of different mechanisms, such as directly calling the patient 602, using a website GUI service 612 (e.g., Merlin.net's DirectCall® phone/messaging service), using the PAM 614, and the like. As a result of stopping the medication, the patient's risk of adverse events, such as bleeding, decreases. Optionally, the system 600 may make automatic prescription changes without clinician intervention.

FIG. 7 is a flow chart for a medical treatment management process 700 for a patient diagnosed with AF. The process 700 may be used in conjunction with the medical treatment management system 500 for AF in FIG. 5 and/or the medical treatment management system 600 for AF in FIG. 6. At 702, a cardiac monitor/sensor shows AF. AF may be recognized by comparing PS data collected by the cardiac monitor 506 (shown in FIG. 5) on the patient 502 (shown in FIG. 5) to PS data that is indicative of AF. Thus, the system 500 in FIG. 5 may diagnose AF with or without the input of a clinician 504 (shown in FIG. 5).

After diagnosis of AF, the process 700 may take chronic management action via a prescription at 704. Non-invasive chronic management may be advisable to treat AF versus an acute intervention. During chronic management 704, the process 700 continues to monitor using the cardiac monitor/sensor at 702 to determine the success of the chronic management. For example, the PS data collected after starting the prescription drug therapy at 704 may indicate that the prescription therapy has not successfully treated the AF, which has since progressed to persistent AF, shown at 706.

Due to the new diagnosis of persistent AF at 706, an acute intervention, such as an ablation, may be recommended and taken at 708. The process 700 may mark the occurrence of the ablation in the patient's PSD history. The cardiac monitor continues to track the patient's PCs related to AF post ablation as it did prior to ablation at 702. At 710, the post ablation monitoring may collect PS data that is processed to reach the conclusion that the ablation procedure has been successful, for example, in treating the patient's AF. As a result of this changed diagnosis, at 712, the prescription therapy used in the chronic management is reduced. Although not shown in FIG. 7, the process 700 may continue after the prescription change. For example, the prescription reduction may be marked in the patient's PSD history, and the cardiac monitor may still continuously monitor for AF. The steps in process 700 may be performed without a clinician's direct involvement. For example, the process 700 may be performed by the system 500, which recommends a course of treatment, while the clinician may use the recommendations to make informed decisions relating to the patient's care management.

FIG. 8 is a diagram showing an example medical treatment management system 800 applied to a patient 802 with chronic and acute hypertension. The medical treatment management system 800 may be the system 200 described in FIG. 2. A patient 802, who may be worried about his/her blood pressure, takes a blood pressure measurement. The patient 802 may take the measurement using an EMD 804, such as a BP cuff. The BP cuff 804 is configured to communicate with intermediary equipment 806, such as through RF signals, induction, or wired connection, to transfer collected PS data relating to BP measurements. The intermediary equipment 806 transmits the BP measurements to a server 808, accessed via a website GUI (such as Merlin.net) for data storage and processing. The patient's BP measurements are viewed online by the patient's physician/clinician 810, who may determine that the patient 802 is hypertensive and should be placed on medication. The physician 810 may use the server/website GUI 810 to contact the patient 802 to schedule an in-clinic visit to discuss the patient's hypertensive condition. The message may be transmitted from the server/website 810 to the intermediary equipment 806, which forwards the message to the patient's PAM 812. At the in-clinic visit, the physician 810 recommends medication to treat the patient's hypertension. The prescribed medication may be, for example, diuretics, beta blockers, calcium channel blockers, ACE inhibitors, and the like.

Upon starting medication, this pivotal medical event is marked and stored in the server 808 as part of the patient's PSD history. The hypertensive patient 802 may be monitored at home using an external BP cuff (sphygmometer) 804 to take regular BP readings to track the effectiveness of the medication therapy, and the PS data collected from the BP cuff is stored in the server 808 as post-medication PS data. The system 800 has a record of the patient's BP measurements and also a record of the patient's prescription regimen through its connection to an external database (e.g., the patient's pharmacy). The system 800 may then recommend to the physician 810 that the patient may be a candidate for renal denervation or implantation of a vagal nerve stimulation device.

The patient 802 has the renal denervation procedure in a cath lab, and the ablation system (not shown) used to perform the procedure sends the procedure type, date, time, and patient info to the server 808 to mark the procedure on a graph and consider it a pivotal medical event (PME). The patient 802 continues home BP monitoring after the procedure, and the patient's PCs, such as blood pressure, continue to be stored by the system 800. The physician 810 reviews the PS data by accessing the patient's BP trend and PMEs timeline through a website 808.

After one month of monitoring after the procedure, the system 800 may have enough post-procedure PS data to make a treatment effectiveness measurement based on the PS data collected before and after the renal denervation acute intervention. For example, the effectiveness measurement may be that the patient's BP has decreased by 25 mm HG systolic and 10 mm Hg diastolic with a confidence of 95%. The system 800 may then recommend that the physician 810 evaluate changing the patient's anti-hypertensive medication regimen now that the patient 802 is normotensive. The physician 810 may then close the loop by instructing the patient 802 to reduce or stop taking the prescribed medication, shown at 814. Optionally, the event of stopping/reducing medication therapy may also be recorded within the server 808 as another pivotal medical event, and the patient's blood pressure measurements may continue to be monitored and stored going forward.

FIG. 9 is a map of a medical treatment management system 900 according to an embodiment that is scalable to integrate various types of data from various sources. The medical treatment management system 900 may be the system 200 shown in FIG. 2. The system 900 has a scalable platform 902 made up of hardware and/or software components, such as the intermediary equipment 220, 222, server 210, and/or website GUI 212 shown in FIG. 2, associated software and circuitry, and other components.

The scalable platform 902 may be operatively connected to multiple sensors 904 that each collect PS data relating to a PC of a patient. The multiple sensors 904 may be implantable devices (IMDs) 906, external devices (EMDs) 908, or a combination of both. Examples of IMDs 906 include pacemakers (i.e., pacers) 910, ICDs 912, implantable cardiac monitors 914, cardiac and spinal cord neurostimulators 916, left atrial pressure (LAP) monitors 918, and deep brain stimulators 920, among others. The EMDs 908 that communicate with the platform 902 may include, for example, BP cuffs 922, external cardiac monitors 924, external event monitors, pulse oximeters, and programmer devices 926 used to program/adjust the IMDs 906. Other EMDs 908, not shown in FIG. 9, may be blood glucometers, weight scales, accelerometers, INR monitors, etc. The various types of sensors 904 listed show that the platform 902 may integrate sensors/devices 904 from multiple medical treatment divisions 928, including cardiac rhythm management 930, atrial fibrillation 932, neuromodulation 934, and/or cardiovascular 936 applications.

The system 900 may use the scalable platform 902 to integrate data from the multiple sensors 904 to treat multiple disease states 938, such as atrial tachycardia/fibrillation (AT/AF) 940, hypertension 942, Parkinsons 944, and chronic pain 946, among others. The treatment of the multiple disease states 938 may be specific to the disease, but management 948 of the multiple disease states 938 include the common elements of monitoring (not shown), diagnosis 950, chronic management 952, and acute management 954. An example of chronic management 952 may be a prescription change, while an acute management 954 example may be a procedure, such as an ablation, implantation of an IMD, or renal denervation.

In addition, the scalable platform 902 may be interoperable with multiple loop closure mechanisms 956, which are communication mechanisms that function to close the loop from diagnosis to treatment. The loop closure mechanisms 956 may be manual 958, such as a clinician manually contacting a patient through a phone call 960, E-mail message 962, fax 964, or text message 966. Alternatively, or in addition, the system 900 may be configured to provide direct messaging 968 to the patient through the same means as above (e.g., phone call 960, E-mail 962, fax 964, or text message 966). Direct messaging 968 may be a communication directly from the system 900 to the patient instead of the communication coming directly from the clinician to the patient, as in manual 958. For example, in direct messaging 968, the clinician may prompt the system 900 to direct message the patient. Furthermore, the loop may be closed through the use of a PAM 970, which may be a device kept proximate to the patient that has a patient user interface. The PAM 970 may be configured to communicate directly with the system 900, and may provide patient notifications and alerts. It should be noted that the multiple loop closure mechanisms 956 may be two-way mechanisms, and therefore may be initiated from the patient to the system 900 and/or clinician, such as the patient manually inputting a subjective pain level using the PAM 970.

FIG. 10 is a diagram showing an example medical treatment management system 1000 applied to a patient 1002 with chronic and acute pain. The medical treatment management system 1000 may be the system 200 described in FIG. 2. The pain may stem from physical trauma or a condition such as migraines, phantom limb pain, diabetic neuropathy, ischemic limb pain, phantom limb pain, failed back surgery, and the like. In this example, a patient 1002 feels back pain so schedules a clinic visit with a physician 1004. The physician 1004 may give a prescription medication and/or recommend a procedure to implant an IMD/attach an EMD 1006 to monitor PC related to pain. The IMD/EMD 1006 may be a neurostimulation device configured with electrode leads that supply neurostimulation (NS) therapy to specific parts of the body to relieve pain. In addition to supplying NS therapy, the NS device, through the electrode leads, also may be used to sense PCs associated with pain, such as heart rate, respiration rate, ST segment changes, etc. Alternatively, or in addition, the EMD 1006 may be an accelerometer designed for tremor control. For example, a patient 1002 with Parkinsons may wear an accelerometer on the wrist that monitors tremors. An external programmer 1007 may be configured to program and re-program/adjust the DBS therapies delivered by the IMD/EMD 1006 externally.

Once the IMD 1006 is implanted (or EMD is attached), the IMD/EMD 1006 collects PS data that is transferred to intermediary equipment 1008 which transmits the data to a server 1010 for storage and processing. For pain management applications, another input may be a patient user interface 1012, such as an IVR phone system, a PAM, and the like, which allows the patient 1002 to enter pain values on a subjective pain scale, which are then uploaded to and stored on the server 1010 (possibly through the intermediary equipment 1008). For example, the patient 10 may use an automated IVR system to input subjective pain ratings on a 1-10 scale daily. The PS data may relate to the PCs associated with pain and may include the subjective pain values. The server 1010 is accessible to the physician 1004 at the clinic, such as via a website GUI. The system 1000 may also be configured to alert the physician 1004. For example, the system 1000 may alert the physician 1004 that the lead impedance of the SCS device 1006 has unexpectedly drifted over the past month, and that the patient's subjective pain ratings have increased. In response to the alert, the physician 1004 may want to bring the patient 1002 into the clinic.

At the clinic, the physician 1004 may, for example, input subjective pain ratings from the day into the system using a computer program that connects to the internet. The physician 1004 reprograms the SCS device 1006 to address the problem(s) by re-programming the device 1006 instead of surgically repositioning the lead. Alternatively, the physician 1004 may determine that surgery is necessary to adjust the location of the leads and/or implant a paddle lead. Each of these actions would be considered a pivotal medical event that would be recorded in the server 1010. After the device 1006 is re-programmed, the SCS device 1006 continues to monitor for pain, and the patient 1002 continues to upload subjective pain values, which detail the post-event pain symptoms. The system 1000 compares the patient's subjective pain ratings in the month before and after device re-programming, and derives an effectiveness indicator. If, for example, the effectiveness indicator indicates that the patient's pain rating is lower now (post-event), the physician 1004 may decide to reduce the patient's prescription and/or modify the stimulation programming. On the other hand, the system 1000 may determine after comparing PS data before and after the re-programming that there is no statistically significant difference in the pain experienced by the patient 1002.

The system 1000 may also treat patients with various neurological conditions, such as dementia, Alzheimers, stroke, concussion, depression, epilepsy, Tourette's syndrome, Parkinsons, obsessive compulsive disorder (OCD), complex regional pain syndrome (CRPS), and even blindness and deafness. For example, for a deaf patient 1002 the system 1000 may include an auditory prosthesis as an EMD that externally processes auditory signals and communicates with the server 1010. Similarly, the system 1000 for a blind patient 1002 may include a visual prosthesis that externally processes visual signals. For patients 1002 with dementia or Alzheimers, the system 1000 may administer cognitive tests to screen for dementia/Alzheimers, to screen for cognitive deficit post-stroke, and/or to adjust medication. The system 1000 may also provide automatic reminders to the patients 1002 to take medication. For a patient 1002 with a concussion, the system 1000 may optionally provide alerts to prevent the patient 1002 from falling asleep.

FIG. 11 illustrates a flowchart of one embodiment of a process 1100 for deriving the effectiveness of medical treatment of a patient. The process 1100 may be implemented by one or more of the system 200, system 300, system 400, system 500, system 600, system 800, system 1000, and the like. At 1102, PS data is collected from one or more IMDs and/or EMDs continuously over a collection interval. For example, the collection interval may be hourly or daily, depending on the type of PS data collected and the use of the PS data to show progressions of the data. The IMDs/EMDs may be sensors configured for use in various health divisions, such as cardiac rhythm monitoring, AF, neuromodulation, and cardiovascular, among others. For example, the collecting step may include collecting first PS data from a first IMD or EMD and collecting second PS data from a second IMD or EMD. The IMDs/EMDs are configured to collect the PS data in relation to one or more PCs of the patient. For example, PCs may include heart rate, ST segment changes, AF burden, blood pressure measurement, and the like.

At 1104, the collected PS data is transferred to a database that is remote from the patient. The PS data may be transferred directly from the IMDs/EMDs, or through intermediary equipment, to a remote server which stores the PS data in the database. Storing the PS data in the database over time forms a PSD history. In an example embodiment, the patient undergoes a pivotal medical event (PME) during the collection period. For example, the PME may represent at least one of a clinical event, an acute intervention, or a prescription change. More specifically, a clinical event may be a heart attack, a stroke, and/or detection of AF, heart failure (HF), or hypertension. Furthermore, examples of an acute intervention may include an ablation, implantation of an ICD device, LAA closure, cardiac resynchronization therapy (CRT) device, SCS therapy, renal denervation, or DBS therapy.

Once the PS data is stored in the database, at 1106, the PS data within the PSD history is analyzed to obtain a proposed treatment therapy (TT). More specifically, the PS data collected prior to the PME may be analyzed in comparison to the PS data collected after the PME to propose a TT at least partially based on the change in the PS data. For example, the TT may be a medication prescription (change), an acute intervention, or to take no affirmative action at this time. The analyzation step at 1106 may be performed autonomously without physician intervention. The proposed TT may be presented to the physician in the form of a recommendation. Optionally, the PS data collected by the IMDs/EMDs at 1102 may be analyzed along with PS data collected by subjective patient input and/or non-patient-specific medical records stored in an external database.

At 1108, the TT may be adjusted based on the latest proposed TT. If the proposed TT is to begin a new treatment therapy, then instead of adjusting the TT, the TT will be implemented for the first time. For example, if the proposed TT is to receive an ablation acute intervention, then at 1108, the patient receives the ablation procedure.

The process 1100 is repeatable. Therefore, after implementing a proposed TT, a determination will be made at 1110 as to whether to return to 1102 to continue collecting PS data from the IMDs/EMDs. Continuing the ablation example, once the patient has received the procedure, flow of the process 1100 moves along the branch denoted by “Y” back to 1102. Therefore, following delivery of the TT, the collecting and transferring operations at 1102 and 1104, respectively, are repeated to obtain post-treatment PS data and form a post-treatment PSD history. Once the process 1100 returns to the analyzation step at 1106, the post-treatment PS data may be analyzed in comparison to the pre-treatment PS data, to propose a TT based on the comparison. Returning to 1108, if the current proposed TT differs from the previous proposed TT, then the TT may be adjusted based on the post-treatment PSD history. Adjusting the TT may constitute a prescription change or an acute intervention. For example, if chronic management of hypertension through prescribed medication has not been successful to treat a patient's hypertension based on the analyzed PS data collected after starting the medication, then the TT may be adjusted by recommending a renal denervation acute intervention. Later, after the renal denervation procedure, if the post-procedure PS data shows that the acute intervention was successful at treating the hypertension, then the TT may be adjusted to reduce the hypertension medication prescription. Another example of an adjustment of the TT may include changing the output of the at least one IMD or EMD. For example, IMDs used to provide neurostimulation or pacing for the heart may be adjusted based on post-treatment PS data.

After adjusting the TT, the process 1100 may periodically skip the repeating step at 1110 and instead move along the branch denoted by “N” to 1112. At 1112, an effectiveness indicator (EI) of the TT is derived based on at least the post-treatment PSD history. Deriving the EI of the TT may include comparing PS data that was collected from at least two different types of IMD or EMD before and after the PME. For example, a system and/or clinician may use the derived EI to determine how effective the TT has been at treating the medical condition/disease. The EI may be recorded in the PSD history that is stored in the database.

After the EI is derived at 1112, flow moves to 1114, where a determination is made whether to repeat the process 1100. The process 1100 may be designed to be continuous and cyclical, such that once at determination step 1114, the process 1100 moves along the branch denoted by “Y” to return to the collection step 1102. However, a physician and/or patient may choose to terminate the process 1100, such as if the patient has been successfully treated and there is no risk of a return of the medical condition/disease state (and the IMDs/EMDs will be removed from the patient). If the determination is made to terminate the process 1100, then flow moves along the branch denote by “N” to a “stop” point at 1116.

FIG. 12 illustrates a flowchart of one embodiment of a process 1200 for deriving the effectiveness of medical treatment of a patient. The process 1200 may be implemented by one or more of the system 200, system 300, system 400, system 500, system 600, system 800, system 1000, and the like. At 1202, first PS data is collected from a first one of an IMD or an EMD over a collection interval. The first IMD or EMD may be configured to collect the first PS data in relation to a first PC of the patient. The first IMD/EMD may be configured to continuously collect the first PS data, even as the flow of the process 1200 moves beyond step 1202. For example, the first IMD/EMD collects PS data before, during, and after a TT is implemented or adjusted in the process 1200.

At 1204, second PS data is collected from a second one of an IMD or an EMD over the collection interval. Like the first IMD/EMD, the second IMD/EMD may be configured to continuously collect the second PS data, including before, during, and after any TT changes. The second IMD or EMD may be configured to collect the second PS data in relation to a second PC of the patient that is different from the first PC. The first IMD/EMD and second IMD/EMD may be configured to treat different first and second diseases, respectively. For example, the first IMD or EMD may represent one of an ICD, pacemaker, SCS device, pressure sensor, or brain stimulator, and the second IMD or EMD may represent one of a blood pressure cuff, external event monitor, programmer device, or a pulse oximeter. Optionally, the first and second PS data may relate to different first and second diseases, or first and second stages of progression of a single disease.

After collecting the first and second PS data at 1202 and 1204, respectively, at 1206, the first and second PS data is transferred to a database which is optionally within or connected to a server. The database is remote from the patient, and the first and second PS data sent to the database forms a PSD history. For example, the database and server may be at a hospital, research institute, data center, and the like. Optionally, the patient may undergo a TT during the collection interval. For example, a TT may include a prescription change and/or an acute intervention. The TT may be recorded within the PSD history.

At 1208, an EI of the TT may be derived by an assessment module based on the first and second PS data collected before and after the TT. During the time immediately after a TT has been implemented or modified, it will take a little time to collect enough post-treatment first and second PS data in order to produce an EI that accurately reflects the effect of the TT on the PCs of the patient. EI values may be stored in the PSD history in the database.

After deriving an EI at 1208, the process moves to 1210 where a TT change may be proposed based on the derived EI. For example, the assessment module may propose (or may be used to propose) a potential prescription change to a user based on the assessment of the effectiveness of the TT. Alternatively, or in addition, the TT change may include coordinating the functional operation of the first IMD/EMD and the second IMD/EMD based on the EI and the first and second PS data. For example, at least one of a sensing parameter, programmed parameter, or an output of the first IMD/EMD may be changed based on the second PS data from the second IMD/EMD. In another example, the assessment module may be configured to set operating parameters of the first IMD/EMD and operating parameters of the second IMD/EMD, both based at least in part on the first PS data and the EI. Once the TT change is proposed, the change may be implemented. Optionally, a clinician may make the decision whether to implement a proposed TT change. The EI may also indicate that the TT should not be changed at the present time, so in response the TT may continue unmodified.

At 1212, a determination is made whether to repeat the process 1200. The process 1200 may be designed to be continuous and cyclical, such that once at determination step 1212, the process 1200 moves along the branch denoted by “Y” to return to the collection step 1202. However, a physician and/or patient may choose to terminate the process 1200. If the determination is made to terminate the process 1200, the flow moves along the branch denote by “N” to a “stop” point at 1214.

For example, the process 1200 in FIG. 12 may be applied to monitor and treat metabolic syndrome and also screen for other diseases, such as diabetes, obesity, and coronary artery disease (CAD). If a patient has one element of metabolic syndrome, the system may start screening for the others. For example, if a patient has insulin resistance and obesity, the system may look for CAD and hypertension by using devices to collect PS data related to elements of these diseases.

To treat diabetes, for example, a patient may be prescribed medication, such as oral insulin, injected insulin, insulin pump, and/or other prescription drug therapies. A glucometer may be the first EMD at 1202 that collects blood glucose measurements from home or lab-based blood sugar tests as the first PS data. A BP cuff may be the second EMD at 1204 that collects blood pressure measurements as the second PS data, which may be used to screen the patient for the development of hypertension over longer periods of time. The glucometer and BP cuff may connect to the server to upload the PS data, as at 1206. The system may monitor patient blood sugar and blood pressure over time from the collected PS data. Additionally, the patient may keep a food diary through a phone app that integrates with the website GUI of the system. The system may derive an effectiveness indicator (EI) based on blood glucose measurements over time, as at 1208, to indicate how successful the prescription medication is treating the diabetes. The EI may show the impact of food and/or exercise on blood sugar over time. The EI may also show the impact of forgetting medication. Over longer periods of time, the system can screen the patient development of hypertension secondary to diabetes. A proposed treatment therapy change at 1210 may be to adjust the chronic prescription therapy or an acute intervention, such as implanting an insulin pump and/or a SCS device for diabetic neuropathy pain. After the TT change, the system may continue to collect blood pressure and blood glucose measurements, returning back to 1202.

To treat obesity, a prescription may be lipase inhibitors, appetite suppressants, and the like. The system may use a scale as a first EMD to collect first PS data that relates to the patient's weight. The second PS data may be body mass index (BMI), and devices that may be used to collect the BMI measurements may be calipers, a water tank test, or a tape measure. The system may include a patient facing tool with internet access for food and exercise logs. The system may provide long term trending and graphing, medication reminders/compliance measurement, create a risk profile for the patient, and suggest screening for other related diseases. Examples of acute interventions that may be proposed as a TT change at 1210 include bariatric surgery, gastric bypass, gastric banding, and neurostimulation for appetite suppression.

For CAD, the first IMD at 1202 may be an ICD, a pacemaker, or an SCS device, and the second IMD/EMD at 1204 may be another one of the three IMDs or an EMD such as a scale, glucometer, or BP cuff. The first and second IMDs/EMDs may monitor ST segment changes, heart rate, and look for signs of obesity and/or diabetes. The system at 1208 may integrate data from a scale, glucometer, and/or BP cuff and evaluate patient risk level along with cardiac data from an ICD/pacemaker. In addition, the system may integrate hospital-based diagnostic data from stress tests, angiography, echo, etc. A proposed TT change at 1210 may be to modify a prescription of nitroglycerin or have an acute intervention, such as an angioplasty, stenting, bypass surgery, or implant another IMD.

One effect of the embodiments described herein is that the system may apply intelligent pattern recognition algorithms to discover treatments that do or do not work for certain types of patients. For example, the algorithms may be applied to data pools gathered within the database by the various inputs, including multiple IMDs/EMDs. The analyzed data may be used for research, and may also lead to the system making treatment therapy recommendations.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the components, arrangements, and configurations described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. A method for deriving effectiveness of medical treatment of a patient, the method comprising:
collecting patient state (PS) data from at least one of an implantable medical device (IMD) or an external medical device (EMD) over a collection interval, the at least one IMD or EMD configured to collect the PS data in relation to a physiologic characteristic (PC) of the patient;
transferring the PS data to a database that is remote from the patient to form a patient state data (PSD) history, wherein the patient undergoes a pivotal medical event (PME) during the collection interval;
analyzing the PS data within the PSD history before and after the PME to propose a treatment therapy (TT);
following delivery of the TT, repeating the collecting and transferring operations to obtain post-treatment PS data and form a post-treatment PSD history; and
deriving an effectiveness indicator (EI) of the TT based on at least the post-treatment PSD history.

2. The method of claim 1, further comprising adjusting the TT based on the post-treatment PSD history.

3. The method of claim 2, wherein adjusting of the TT constitutes a prescription change or an acute intervention.

4. The method of claim 2, wherein the adjusting of the TT constitutes changing an output the at least one IMD or EMD.

5. The method of claim 1, wherein the deriving the effectiveness of the TT comprises comparing PS data that was collected from at least two different types of IMD or EMD before and after the PME.

6. The method of claim 1, wherein the collecting includes collecting first PS data from a first IMD or EMD and collecting second PS data from a second IMD or EMD.

7. The method of claim 1, wherein the PME represents at least one of a clinical event, an acute intervention, or a prescription change.

8. The method of claim 7, wherein the acute intervention comprises an ablation, implantation of an implantable cardioverter-defibrillator (ICD) device, left atrial appendage (LAA) closure, cardiac resynchronization therapy (CRT), spinal cord stimulation (SCS) therapy, renal denervation, or deep brain stimulation (DBS) therapy.

9. The method of claim 7 wherein the clinical event comprises at least one of a heart attack, a stroke, or detection of atrial fibrillation (AF), heart failure, or hypertension.

10. A method for deriving effectiveness of medical treatment of a patient, the method comprising:
collecting first patient state (PS) data from a first implantable medical device (IMD) over a collection interval, the first IMD configured to collect the first PS data in relation to a first physiologic characteristic (PC) of the patient;
collecting second PS data from a second IMD, the second IMD configured to collect the second PS data in relation to a second PC of the patient, the first PC differing from the second PC;
transferring the first and second PS data to a database that is remote from the patient to form a patient state data (PSD) history, wherein the patient undergoes a treatment therapy (TT) during the collection interval;
deriving an effectiveness indicator (EI) of the TT, by an assessment module, based on the first and second PS data collected before and after the TT.

11. The method of claim 10, further comprising proposing, by the assessment module, a potential prescription change to a user based on the assessment of the effectiveness.

12. The method of claim 10, further comprising changing at least one of a sensing, programmed parameters, or an output of the first IMD based on the second PS data from the second IMD.

13. The method of claim 10, further comprising integrating the first and second PS data where the first and second PS data relate to at least one of i) different first and second diseases or ii) first and second stages of progression of a single disease.

14. The method of claim 10, further comprising coordinating functional operation of the first IMD and the second IMD based on the EI and the first and second PS data.

15. The method of claim 10, wherein the first IMD and the second IMD are configured to treat different first and second diseases, respectively, the method further comprising setting, at the assessment module, operating parameters of the first IMD and operating parameters of the second IMD both based at least in part on the first PS data and the EI.

16. The method of claim 10, wherein the first IMD represents one of an ICD, pacemaker, spinal cord stimulation device, pressure sensor, or brain stimulator.

17. A system for deriving effectiveness of medical treatment of a patient, comprising:
at least one of an implantable medical device (IMD) or an external medical device (EMD) for collecting patient state (PS) data from over a collection interval, the at least one IMD or EMD configured to collect the PS data in relation to a physiologic characteristic (PC) of the patient;
a database configured to receive the PS data to form a patient state data (PSD) history, the database being remote from the patient, wherein the patient undergoes a pivotal medical event (PME) during the collection interval;
an analysis module configured to analyze the PS data within the PSD history before and after the PME to propose a treatment therapy (TT);
following delivery of the TT, the IMD or EMD and database repeating the collecting and receiving operations to obtain post-treatment PS data and a post-treatment PSD history; and
an assessment module configured to derive an effectiveness indicator (EI) of the TT based on at least the post-treatment PSD history.

18. The system of claim 17, further comprising a TT control module configured to adjust the treatment therapy based on the post-treatment PSD history.

19. A system for deriving effectiveness of medical treatment of a patient, the system comprising:
a first implantable medical device (IMD) configured to collect first patient state (PS) data over a collection interval, the first IMD configured to collect the first PS data in relation to a first physiologic characteristic (PC) of the patient;
a second IMD configured to collect second PS data in relation to a second PC of the patient, the first PC differing from the second PC;
a database located remote from the patient, the database configured to receive the first and second PS data to form a patient state data (PSD) history, wherein the patient undergoes a treatment therapy (TT) during the collection interval; and
an assessment module configured to derive an effectiveness indicator (EI) of the TT based on the first and second PS data collected before and after the TT.

20. The system of claim 19, wherein the assessment module is configured to propose a potential prescription change to a user based on the assessment of the effectiveness of the TT.
